(19) United States
(12) Patent Application Publication
(10) Pub. No.: US 2025/0367101 A1
(43) Pub. Date: Dec. 4, 2025

(54) PERSONAL CARE COMPOSITION

(71) Applicant: THE PROCTER & GAMBLE COMPANY, Cincinnati, OH (US)

(72) Inventors: Jessa Leigh MEYERS, Cincinnati, OH (US); Brennan Alexander SCHILLING, Cincinnati, OH (US); Eric Scott JOHNSON, Hamilton, OH (US); Parth Rajesh SHAH, Mason, OH (US)

(21) Appl. No.: 19/227,702

(22) Filed: Jun. 4, 2025

Related U.S. Application Data
(60) Provisional application No. 63/655,887, filed on Jun. 4, 2024.

(51) Int. Cl. A61K 8/73; A61K 8/44; A61K 8/46; A61K 8/891; A61Q 5/12

(52) U.S. Cl. CPC A61K 8/73; A61K 8/44; A61K 8/463; A61K 8/466; A61K 8/737; A61K 8/891; A61Q 5/12

(57) ABSTRACT

A personal care composition that includes a biodegradable conditioning agent and has provides good stability, deposition and conditioning. The composition includes a cationically modified poly alpha-1,6-glucan ether compound, an anionic detersive surfactant, a co-surfactant and an aqueous carrier.

### FIELD

The invention relates, generally, to personal care compositions comprising a biodegradable, low-molecular weight, cationic polymer. More specifically, the invention relates to a personal care composition comprising a low molecular weight cationic modified poly alpha-1,6-glucan ether compound, which provides good hair conditioning, composition stability and silicone deposition.

### BACKGROUND

Shampoos, conditioners and body washes, collectively referred to as personal care compositions, are well known for use in cleaning skin and hair. These compositions typically include a detersive surfactant that helps solubilize dirt, oil and other contaminants, which can then be rinsed away with water. Anionic sulfated surfactants such as sodium lauryl sulfate may be particularly preferred for use as detersive surfactants because they provide good lather, good cleansing and can be paired with cationic polymers used provide a conditioning benefit.

Another advantage of sulfated surfactants is they generally do not interfere with the viscosity modifying agents (e.g., salt) present in the composition. Viscosity can be important in a personal care composition, especially in a shampoo. Consumers may perceive a shampoo that is too thin as being poor quality, whereas a shampoo that is too thick may not spread easily enough. Personal care compositions are generally formulated to have a viscosity that enables convenient application of the composition to a target surface (e.g., hair or scalp), for example, by dispensing the composition into an open palm and then spreading it across the target surface.

Recently, however, sulfated surfactants have been perceived as being harsh on hair. That is, some consumers believe that sulfated surfactants strip away the natural oils produced by the scalp to help protect hair, which leaves hair dry and dull looking. Thus, it would be desirable to provide a shampoo with a sulfate-free surfactant system that provides good in-use benefits (lather, spreadability, cleansing, etc.).

While shampoos are generally good at removing dirt, oil and other contaminants from hair, they can have some drawbacks such as, for example, hair frizz. Hair frizz is described by consumers as the appearance of unruly fibers at the top of the scalp and tips of hair as well as an increased volume through the bulk of the hair, and it can be especially noticeable on days when there is humid weather and the level of moisture in the air is high. Of course, when the air is dry, frizz may be present as a result of electrostatic repulsion between the hair fibers, sometimes referred to as “static.” A common strategy to prevent hair frizz is to deposit a conditioning agent (e.g., silicone, oil or cationic polymers polymer) onto the surface of the hair to make it more hydrophobic, thereby decreasing inter-fiber interactions. When applied at a suitably high level, the conditioning agent increases the cohesive forces holding the hair fibers together to prevent frizz from occurring. However, when applied at such levels, conventional conditioning agents (e.g., polyquaterniums and silicones) and can build up on hair resulting in a heavy, sticky or greasy look and/or feel. Thus, it would be desirable to provide a conditioning agent that provides good frizz control and minimizing the undesirable look and feel drawbacks of conventional conditioning agents.

In addition to providing frizz control, conditioning compositions can also address other drawbacks sometimes attributed to shampoo use. For example, some consumers have indicated that shampoos make their hair: more prone to tangling (wet and dry), harder to comb and/or style, look and feel dry, look and feel rough, and/or look damaged (e.g., “split ends”). Conditioning compositions may help alleviate some of these undesirable hair issues by depositing a conditioning agent onto hair. The conditioning agent forms a film on the hair shaft to help hold in moisture and smoothen the surface of the hair Thus, in some instances, it may be desirable to formulate a personal care composition that provides cleansing and conditioning benefits, for example, by including a detersive surfactant and a conditioning agent. Such products are sometimes referred to as conditioning shampoos or 2-in-1 shampoos.

In some instances, conditioning shampoos work by forming a coacervate during use, when the composition is diluted with water. However, formulating a shampoo containing a sulfate-free anionic surfactant and a cationic conditioning polymer can be difficult because they tend to form an in situ coacervate that can cause formulation instability and/or inconsistent in-use performance. Product instability can manifest as an undesirably cloudy appearance and/or the presence of a precipitate layer, which may be associated with poor product quality by consumers. Thus, it is desirable for these conditioning shampoos to be isotropic, and thus not have an in situ coacervate present in the composition prior to use (rather than during use, which is desired).

### SUMMARY

Cationic polymers are known for use as conditioning agents. However, many of these cationic polymers are non-biodegradable (e.g., silicones). Due to growing social concerns about environmental responsibility, the use of silicone conditioning agents has fallen out of favor for some consumers. While a biodegradable conditioning system may be desirable for some consumers, it has been found that biodegradable polymers that are suitable for use as conditioning agents tend to be salt intolerant, which is undesirable when inorganic salt (e.g., NaCl) is used as a thickener in the composition, and/or do not perform as well as their non-biodegradable counterparts. Accordingly, it would be desirable to provide a conditioner comprising a biodegradable cationic conditioning system that exhibits good salt tolerance and conditioning performance.

### BRIEF DESCRIPTION OF THE DRAWINGS

FIG. 1 illustrates the conditioning benefits of the cationic poly alpha-1,6-glucan ether compounds compared to conventional cationic conditioning polymers.

FIG. 2 illustrates the silicone deposition benefit of the inventive personal care compositions.

FIGS. 3A and 3B illustrate the dilute coacervate benefit of a combination of guar hydoxypropyltrimonium chloride cationic polymer and a poly alpha-1,6-glucan ether compound.

### DETAILED DESCRIPTION

Polymer biodegradability is generally inversely proportional to polymer molecular weight. However, higher molecular weight cationic polymers provide better conditioning than their lower molecular weight counterparts. Thus, providing a conditioning shampoo with a suitable biodegradable cationic conditioning agent has long been a challenge because the lower molecular weight polymers do not perform as well. For example, experiments have shown that polymers with a molecular weight of less than 400 kDa do not form a suitable coacervate when diluted with water or sufficiently adsorb to hair. For example, polyquaternium-10 polymers such as UCare® Polymer FP (MW of 120,000 and Charge density of 0.7 meq/g) from Dow/Amerchol or guar hydroxypropyltrimonium chloride polymer such as AquaCat CG518 (MW<400,000 and charge density of 0.7 meq/g) from Ashland do not provide suitable conditioning or stability.

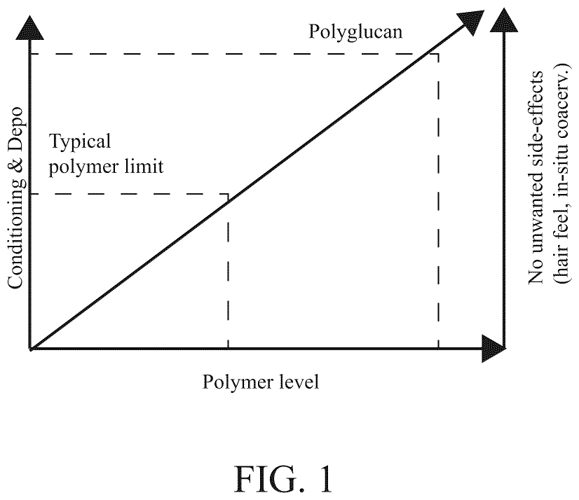

Surprisingly, it has now been found that cationic poly alpha-1,6-glucan ether compounds (INCI: alpha-glucan hydroxypropyltrimonium chloride) with a molecular weight of less than 500 kDa (e.g., 50 kDa to 400 kDa) exhibit good biodegradability and conditioning benefits. This is unexpected because it is generally believed that cationic conditioning polymers with a molecular weight of less than 500 kDa do not provide suitable conditioning. Hence, cationic polymers such as guar hydroxypropyltrimonium chloride (e.g., Jaguar Excel® available from Syensquo and N-hance 3196 available from Ashland), with a molecular weight of approximately 1.1 MDa, are more commonly used as conditioning agents. FIG. 1 illustrates the surprising conditioning benefits provided by the cationic poly alpha-1,6-glucan ether compounds according to the present disclosure.

Reference to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Alkyl” means a saturated linear, branched, aralkyl (such as benzyl), or cyclic (“cycloalkyl”) hydrocarbon group, and includes substituted alkyls (e.g., hydroxyalkyl substituents or dihydroxy alkyl substituents), as well as alkyl groups containing one or more heteroatoms such as oxygen, sulfur, and/or nitrogen within the hydrocarbon chain.

“Aryl” means an aromatic carbocyclic group having a single ring (e.g., phenyl), multiple rings (e.g., biphenyl), or multiple condensed rings in which at least one is aromatic, (e.g., 1,2,3,4 tetrahydronaphthyl, naphthyl, anthryl, or phenanthryl), which is optionally mono, di, or trisubstituted with alkyl groups. Aryl also includes heteroaryl groups where heteroaryl is defined as 5, 6, or 7 membered aromatic ring systems having at least one hetero atom selected from the group consisting of nitrogen, oxygen and sulfur. Examples of heteroaryl groups include pyridyl, pyrimidinyl, pyrrolyl, pyrazolyl, pyrazinyl, pyridazinyl, oxazolyl, furanyl, imidazole, quinolinyl, isoquinolinyl, thiazolyl, and thienyl, which can optionally be substituted with alkyl groups.

“Apply” or “application” means to apply or spread the composition onto a human keratinous surface such as skin or hair.

“Biodegradable” means a material or product that is totally utilized by microorganisms resulting in the production of carbon dioxide, water, mineral salts and new microbial cellular constituents (biomass).

“Clear” means that a composition has percent transmittance (% T) of at least 80% at 600 nm. Percent transmittance can be determined according to the method described in more detail below.

“Conditioning agent” means any substance, as well as any component thereof, that is intended to provide a conditioning benefit (e.g., deposition of active ingredients, improved wet feel, reduced hair friction, wet or dry detangling, reduced wet or dry combing force, or increased smoothness, shine or volume).

“Charge density” (“CD”) means the ratio of positive charges on a polymer to the molecular weight of the polymer.

“Degree of substitution” (“DoS”) means the average number of substituted groups per glucose monomer. DoS can be determined according to conventional method known in the art (e.g., nuclear magnetic resonance (NMR)).

“Molecular weight” (“Mw”) means weight average molecular weight and can be determined using various techniques known in the art such as high-pressure liquid chromatography (HPLC), size exclusion chromatography (SEC), gel permeation chromatography (GPC), and gel filtration chromatography (GFC). Molecular weight is reported in daltons (Da).

“Personal care composition” refers to a composition or product intended for use in cleaning or conditioning skin and/or hair. Some non-limiting examples of personal care compositions are shampoos, conditioners, conditioning shampoos, body washes, shower gels, liquid hand cleansers, facial cleansers, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

### Personal Care Composition

The personal care compositions herein include a surfactant system that includes a detersive anionic surfactant and a conditioning system. The surfactant system includes an anionic detersive surfactant and may include one or more co-surfactants. The conditioning system may be present at 0.1% to 5% (e.g., 0.25% to 4%, 0.5% to 3% or 0.75% to 2%), based on the weight of the composition, and may include other cationic polymers and/or conditioning agents such as cationic silicone polymers and polyquaternium compounds. The personal care composition includes 50% to 90% of an aqueous carrier, which may be entirely water or a mixture of water and other water-miscible solvents. The personal care composition may, optionally, include other ingredients commonly used in personal care compositions.

In some instances, the personal care compositions may be free of sulfated surfactants, or substantially free of them. For sulfate-free embodiments, it may also be desirable to limit the amount of inorganic salt (e.g., NaCl, KCL, CaCl, MgCl, NaSO4) present in the composition to less than 1% (e.g., less than 0.75%, 0.5% or even less than 0.25%). Low-salt, sulfate-free compositions have been found to be more stable than those containing higher levels of inorganic salt.

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the dermatologically acceptable carrier chosen. In some aspects, the personal care compositions described herein may include a dispersed gel network that provides a conditioning benefit to hair.

The personal care composition may have a pH of greater than 3.0 (e.g., 4.0 to 10, 4.5 to 8, or 5 to 6.5). The personal care compositions are liquids and can be Newtonian or non-Newtonian. The liquid personal care compositions have a viscosity of 500 mPa·s to 30,000 mPa·s, (e.g., 100 mPa-s to 20,000 mPa·s, 2000 mPa-s to 15,000 mPa·s, or 5000 mPa·s to 12,000 mPa·s).

In some aspects, the personal care compositions can be clear. For example, the personal care composition may have a % T of 80% to 100%, 85% to 100%, 90% to 100% or even 95% to 100%).

### Surfactant System

The surfactant system may be present at 5% to 50% (e.g., 15% to 40% or 20%-35%), based on the weight of the composition. The surfactant system includes an anionic detersive surfactant and at least one co-surfactant selected from non-ionic surfactants, amphoteric surfactants and zwitterionic surfactants.

### Anionic Surfactant

Some nonlimiting examples of anionic surfactants that may be suitable for use herein are alkyl sulfates; alkyl ether sulfates; acyl glycinates; acyl sarcosinates; acyl glutamates; acyl alaninates; sulfosuccinates, isethionates; sulfonates; sulfoacetates; glucose carboxylates; alkyl ether carboxylates; acyl taurates; sodium, ammonium or potassium salts of these; and combinations thereof. In some instances, the alkyl sulfate anionic surfactant can alkoxylated with an average degree of alkoxylation of less than 3.5 (e.g., 0.3 to 2.0 or 0.5 to 0.9), which is believed to help improve low temperature physical stability and suds mileage of the composition. Methods for determining degree of alkoxylation are known in the art, for example, as described in US 2023/0045856.

Examples of anionic sulfate surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine, lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate and sodium cocoyl isethionate. Sodium lauryl sulfate or sodium laureth sulfate may be particularly suitable.

Examples of sulfosuccinate surfactants include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid and dioctyl esters of sodium sulfosuccinic acid.

Examples of isethionate surfactants include sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate and sodium stearoyl methyl isethionate.

Examples of sulfonates include alpha olefin sulfonates (e.g., C14-16 alpha olefin sulfonate), linear alkylbenzene sulfonates and sodium laurylglucosides hydroxypropylsulfonate.

Examples of sulfoacetates include sodium lauryl sulfoacetate and ammonium lauryl sulfoacetate.

Example of glucose carboxylates include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate and sodium C12-15 pareth-8 carboxylate.

Examples of acyl taurates include sodium methyl cocoyl taurate, sodium cocoyl taurate, sodium methyl lauroyl taurate, sodium lauroyl taurate and sodium methyl oleoyl taurate.

### Co-Surfactant

The surfactant system herein may include 5% to 50% of a co-surfactant, based on the weight of the surfactant system and/or 1% to 15% (e.g., 2-10%, 3-9%, 4-8%, or even 5-7%), based on the weight of the composition. The amount of co-surfactant in the composition can be important and should be tailored to balance solubility and/or viscosity building with cleaning and/or conditioning benefit. For example, too much amphoteric co-surfactant can make the surfactant system less salt tolerant and may impede the ability of the surfactant system to form a suitable coacervate upon dilution with water. This can be especially problematic when the composition contains a cationic polymer because the lowered salt tolerance of the surfactant system may cause the cationic polymer to precipitate out. In some embodiments, the co-surfactant may be present at a weight ratio of detersive surfactant to co-surfactant of 12:1 to 3:10 (6:1 to 3:10, 4:1 to 1:3, or even 2:1 to 1:2).

Some non-limiting examples of amphoteric and zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, hydroxysultaines, amidopropyl hydroxysultaines, and combinations thereof. Some non-limiting examples of amphoteric surfactants include amphoacetates, amphodiacetates, betaines, amidobetaines (e.g., cocamidopropyl betaine and lauramidopropyl betaine), propionates, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereolf. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and Mccutcheon's Emulsifiers and Detergents, 2019, MC Publishing Co.

### Cationically Modified Poly Alpha-1,6-Glucan Ether Compound

The personal care compositions herein include a cationically modified poly alpha-1,6-glucan ether compound. In some aspects, the cationically modified poly alpha-1,6-glucan ether compound contains a poly alpha-1,6-glucan substituted with at least one positively charged organic group, wherein the poly alpha-1,6-glucan comprises a backbone of glucose monomer units where at least 65% of the glucose monomer units are linked via alpha-1,6-glycosidic linkages, and where the poly alpha-1,6-glucan ether compound has a degree of substitution of about 0.001 to about 3; and is characterized by a weight average molecular weight of from about 1000 to about 500,000 daltons and/or being derived from a poly alpha-1,6-glucan having a weight average molecular weight of from about 900 to about 450,000 daltons, determined prior to substitution with the least one positively charged organic group.

Glucose carbon positions 1, 2, 3, 4, 5 and 6 as referred to herein are as known in the art and depicted in Structure I:

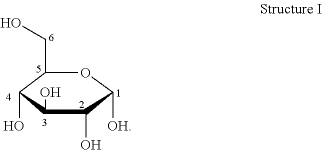

The terms “glycosidic linkage” and “glycosidic bond” are used interchangeably herein and refer to the type of covalent bond that joins a carbohydrate (sugar) molecule to another group such as another carbohydrate. The term “alpha-1,6-glucosidic linkage” as used herein refers to the covalent bond that joins alpha-D-glucose molecules to each other through carbons 1 and 6 on adjacent alpha-D-glucose rings. The term “alpha-1,3-glucosidic linkage” as used herein refers to the covalent bond that joins alpha-D-glucose molecules to each other through carbons 1 and 3 on adjacent alpha-D-glucose rings. The term “alpha-1,2-glucosidic linkage” as used herein refers to the covalent bond that joins alpha-D-glucose molecules to each other through carbons 1 and 2 on adjacent alpha-D-glucose rings. The term “alpha-1,4-glucosidic linkage” as used herein refers to the covalent bond that joins alpha-D-glucose molecules to each other through carbons 1 and 4 on adjacent alpha-D-glucose rings. Herein, “alpha-D-glucose” will be referred to as “glucose”.

The glycosidic linkage profile of a glucan, dextran, substituted glucan, or substituted dextran can be determined using any suitable method known in the art. For example, a linkage profile can be determined using methods that use nuclear magnetic resonance (NMR) spectroscopy (e.g., 13C NMR or 1H NMR). These and other methods that can be used are disclosed in Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The structure, molecular weight, and degree of substitution of a polysaccharide or polysaccharide derivative can be confirmed using various physiochemical analyses known in the art such as NMR spectroscopy and size exclusion chromatography (SEC).

The poly alpha-1,6-glucan ether compound comprises a poly alpha-1,6-glucan substituted with at least one positively charged organic group, where the poly alpha-1,6-glucan comprises a backbone of glucose monomer units, where at least 65% of the glucose monomer units are linked via alpha-1,6-glycosidic linkages. The poly alpha-1,6-glucan ether compound may be characterized by (a) a weight average degree of polymerization of at least 5; (b) a weight average molecular weight of from about 1000 to about 500,000 daltons; and/or (c) having been derived from a poly alpha-1,6-glucan having a weight average molecular weight of from about 900 to about 450,000 daltons, determined prior to substitution with the least one positively charged organic group. The poly alpha-1,6-glucan ether compound may be characterized by a degree of substitution of about 0.001 to about 3.0. Optionally, at least 3%, preferably from about 5% to about 50%, more preferably from about 5% to about 35%, of the backbone glucose monomer units have branches via alpha-1,2 and/or alpha-1,3-glycosidic linkages. These compounds, groups, and properties are described in more detail below.

The poly alpha-1,6-glucan ether compounds disclosed herein comprise poly alpha-1,6-glucan substituted with at least one positively charged organic group, wherein the organic group or groups are independently linked to the poly alpha-1,6-glucan polysaccharide backbone and/or to any branches, if present, through an ether (—O—) linkage. The at least one positively charged organic group can derivatize the poly alpha-1,6-glucan at the 2, 3, and/or 4 glucose carbon position(s) of a glucose monomer on the backbone of the glucan, and/or at the 1, 2, 3, 4, or 6 glucose carbon position(s) of a glucose monomer on a branch, if present. At unsubstituted positions a hydroxyl group is present in a glucose monomer.

The poly alpha-1,6-glucan ether compounds disclosed herein are referred to as cationic ether compounds due to the presence of one or more positively charged organic groups. The terms “positively charged organic group”, “positively charged ionic group”, and “cationic group” are used interchangeably herein. A positively charged group comprises a cation (a positively charged ion). Examples of positively charged groups include substituted ammonium groups, carbocation groups, and acyl cation groups.

The cationic poly alpha-1,6-glucan ether compounds disclosed herein comprise water-soluble poly alpha-1,6-glucan comprising a backbone of glucose monomer units wherein at least 65% of the glucose monomer units are linked via alpha-1,6-glycosidic linkages, and optionally at least 5% of the backbone glucose monomer units have branches via alpha-1,2 and/or alpha-1,3-glycosidic linkages. The poly alpha-1,6-glucan is substituted with positively charged organic groups on the polysaccharide backbone and/or on any branches which may be present, such that the poly alpha-1,6-glucan ether compound comprises unsubstituted and substituted alpha-D-glucose rings. The poly alpha-1,6-glucan may be randomly substituted with positively charged organic groups. As used herein, the term “randomly substituted” means the substituents on the glucose rings in the randomly substituted polysaccharide occur in a non-repeating or random fashion. That is, the substitution on a substituted glucose ring may be the same or different (i.e., the substituents, which may be the same or different, on different atoms in the glucose rings in the polysaccharide) from the substitution on a second substituted glucose ring in the polysaccharide, such that the overall substitution on the polymer has no pattern. Further, the substituted glucose rings may occur randomly within the polysaccharide (i.e., there is no pattern with the substituted and unsubstituted glucose rings within the polysaccharide).

Depending on reaction conditions and the specific substituent used to derivatize the poly alpha-1,6-glucan, the glucose monomers of the polymer backbone may be disproportionately substituted relative to the glucose monomers of any branches, including branches via alpha-1,2 and/or alpha-1,3 linkages, if present. The glucose monomers of the branches, including branches via alpha-1,2 and/or alpha-1,3 linkages, if present, may be disproportionately substituted relative to the glucose monomers of the polymer backbone. Depending on reaction conditions and the specific substituent used, substitution of the poly alpha-1,6-glucan may occur in a block manner.

Depending on reaction conditions and the specific substituent used to derivatize the poly alpha-1,6-glucan, it is possible that the hydroxyl groups at certain glucose carbon positions may be disproportionately substituted. For example, the hydroxyl at carbon position 6 for a branched unit may be more substituted than the hydroxyls at other carbon positions. The hydroxyl at carbon position 2, 3, or 4 may be more substituted than the hydroxyls at other carbon positions.

The poly alpha-1,6-glucan ether compounds disclosed herein contain positively charged organic groups and are of interest due to their solubility characteristics in water, which can be varied by appropriate selection of substituents and the degree of substitution. The poly alpha-1,6-glucan ether compound may have a DoS of about 0.001 to about 1.5 and a solubility of 0.1% by weight or higher in deionized water at 25° C. The poly alpha-1,6-glucan ether compound may have a DoS of about 0.05 to about 1.5 and a solubility of less than 0.1% by weight in pH 7 water at 25° C. Poly alpha-1,6-glucan ether compounds having a solubility of at least 0.1%, or at least 1%, or at least 10%, or at least 25%, or at least 50%, or at least 75%, or at least 90%, by weight, in deionized water at 25° C. may be preferred for use in fabric care or dish care compositions, due to ease of processing and/or increased solubility in aqueous end-use conditions.

The poly alpha-1,6-glucan ether compounds may include a substituted poly alpha-1,6-glucan, and are typically made from a poly alpha-1,6-glucan starting material. The terms “poly alpha-1,6-glucan” and “dextran” are used interchangeably herein. Dextrans represent a family of complex, branched alpha-glucans generally comprising chains of alpha-1,6-linked glucose monomers, with periodic side chains (branches) linked to the straight chains by alpha-1,3-linkage (Ioan et al., Macromolecules 33:5730-5739) or alpha-1,2-linkage. Production of dextrans is typically done through fermentation of sucrose with bacteria (e.g., Leuconostoc or Streptococcus species), where sucrose serves as the source of glucose for dextran polymerization (Naessens et al., J. Chem. Technol. Biotechnol. 80:845-860; Sarwat et al., Int. J. Biol. Sci. 4:379-386; Onilude et al., Int. Food Res. J. 20:1645-1651). Poly alpha-1,6-glucan can be prepared using glucosyltransferases such as those described in WO2015/183714 and WO2017/091533.

The cationic poly alpha-1,6-glucan ether compound may comprise a backbone of glucose monomer units wherein greater than or equal to 40% of the glucose monomer units are linked via alpha-1,6-glycosodic linkages, for example greater than or equal to 40%, 45%, 50%, 55%, 60%, 65%, 70%, 75%, 80%, 90%, or 95% of the glucose monomer units. The backbone of the cationic poly alpha-1,6-glucan ether compound can include at least 3%, 5%, 10%, 15%, 20%, 25%, 30%, 35%, 40%, 45%, 50%, 55%, or 60% glucose monomer units which are linked via alpha-1,2, alpha-1,3, and/or alpha-1,4 glycosidic linkages. The cationic poly alpha-1,6-glucan ether compound may include a backbone of glucose monomer units wherein at least 65% of the glucose monomer units are linked via alpha-1,6-glycosidic linkages. The cationic poly alpha-1,6-glucan ether compound may include a backbone of glucose monomer units wherein at least 70% of the glucose monomer units are linked via alpha-1,6-glycosidic linkages. The cationic poly alpha-1,6-glucan ether compound may include a backbone of glucose monomer units wherein at least 80% of the glucose monomer units are linked via alpha-1,6-glycosidic linkages. The cationic poly alpha-1,6-glucan ether compound may include a backbone of glucose monomer units wherein at least 90% of the glucose monomer units are linked via alpha-1,6-glycosidic linkages. The cationic poly alpha-1,6-glucan ether compound may include a backbone of glucose monomer units wherein at least 95% of the glucose monomer units are linked via alpha-1,6-glycosidic linkages. The cationic poly alpha-1,6-glucan ether compound may include a backbone of glucose monomer units wherein at least 99.5% of the glucose monomer units are linked via alpha-1,6-glycosidic linkages. The poly alpha-1,6-glucan ether compound may be predominantly linear.

Dextran “long chains” can include “substantially (or mostly) alpha-1,6-glucosidic linkages”, meaning that they can have at least about 98.0% alpha-1,6-glucosidic linkages in some aspects. Dextran herein can include a “branching structure” (branched structure) in some aspects. It is contemplated that in this structure, long chains branch from other long chains, likely in an iterative manner (e.g., a long chain can be a branch from another long chain, which in turn can itself be a branch from another long chain, and so on). It is contemplated that long chains in this structure can be “similar in length”, meaning that the length (e.g., measured by DP/degree of polymerization) of at least 70% of all the long chains in a branching structure is within plus/minus 30% of the mean length of all the long chains of the branching structure.

Dextran may further include “short chains” branching from the polysaccharide backbone, the branches typically being one to three glucose monomers in length, and typically comprising less than about 10% of all the glucose monomers of a dextran polymer. Such short chains typically include alpha-1,2-, alpha-1,3-, and/or alpha-1,4-glucosidic linkages (it is understood that there can also be a small percentage of such non-alpha-1,6 linkages in long chains in some aspects). The amount of alpha-1,2-branching or alpha-1,3-branching can be determined by NMR methods, as disclosed in the Test Methods.

Dextran can be produced enzymatically prior to being modified with alpha-1,2 or alpha-1,3 branches. In certain embodiments, dextran can be synthesized using a dextransucrase and/or methodology as disclosed in WO 2015/183714 or WO 2017/091533 or published application US 2018/0282385, which are all incorporated herein by reference. The dextransucrase identified in these references can be used, if desired (or any dextransucrase comprising an amino acid sequence that is at least about 90%, 91%, 92%, 93%, 94%, 95%, 96%, 97%, 98%, or 99% identical to any of these particular dextransucrases). Such enzymatically produced dextran is linear (i.e., 100% alpha-1,6-linkages) and aqueous soluble.

The poly-1,6-glucan with branching can also be produced enzymatically using methods known in the art. Without wishing to be bound by theory, it is believed that branching can increase the solubility of the poly alph-1,6-glucan ether compound, which can lead to more convenient processability and/or transport. It is also believed that limits on the degree of branching can lead to improved performance in the final treatment composition.

A poly alpha-1,6-glucan ether compound may have a degree of alpha-1,2-branching that is less than 50%. A poly alpha-1,6-glucan ether compound may have a degree of alpha-1,2-branching that is at least 5%. From about 5% to about 50% of the backbone glucose monomer units of a poly alpha-1,6-glucan ether compound may have branches via alpha-1,2 or alpha-1,3 glycosidic linkages. From about 5% to about 35% of the backbone glucose monomer units of a poly alpha-1,6-glucan ether compound may have branches via alpha-1,2 or alpha-1,3 glycosidic linkages.

At least about 3%, preferably at least about 5% of the backbone glucose monomer units of a poly alpha-1,6-glucan ether compound may have branches via alpha-1,2- or alpha-1,3-glycosidic linkages. A poly alpha-1,6-glucan ether compound may include a backbone of glucose monomer units wherein greater than or equal to 65% of the glucose monomer units are linked via alpha-1,6-glycosidic linkages. A poly alpha-1,6-glucan ether compound may include a backbone of glucose monomer units wherein greater than or equal to 65% of the glucose monomer units are linked via alpha-1,6-glycosidic linkages and at least3%, preferably at least 5%, preferably from about 5% to about 30%, more preferably from about 5% to about 25%, even more preferably from about 5% to about 20%, of the glucose monomer units have branches via alpha-1,2- or alpha-1,3-glycosidic linkages. A poly alpha-1,6-glucan ether compound may include a backbone of glucose monomer units wherein greater than or equal to 65% of the glucose monomer units are linked via alpha-1,6-glycosidic linkages and at least 5% of the glucose monomer units have branches via alpha-1,2 linkages. A poly alpha-1,6-glucan ether compound may include a backbone of glucose monomer units wherein greater than or equal to 65% of the glucose monomer units are linked via alpha-1,6-glycosidic linkages and at least 5% of the glucose monomer units have branches via alpha-1,3 linkages. A poly alpha-1,6-glucan ether compound may include a backbone of glucose monomer units wherein greater than or equal to 65% of the glucose monomer units are linked via alpha-1,6-glycosidic linkages and from about 5% to about 50% of the glucose monomer units have branches via alpha-1,2- or alpha-1,3-glycosidic linkages. A poly alpha-1,6-glucan ether compound may include a backbone of glucose monomer units wherein greater than or equal to 70% of the glucose monomer units are linked via alpha-1,6-glycosidic linkages and from about 5% to about 35% of the glucose monomer units have branches via alpha-1,2- or alpha-1,3-glycosidic linkages.

A poly alpha-1,6-glucan ether compound may include a backbone of glucose monomer units wherein greater than or equal to 90% of the glucose monomer units are linked via alpha-1,6-glycosidic linkages. A poly alpha-1,6-glucan ether compound may include a backbone of glucose monomer units wherein greater than or equal to 90% of the glucose monomer units are linked via alpha-1,6-glycosidic linkages and at least 5% of the glucose monomer units have branches via alpha-1,2- or alpha-1,3-glycosidic linkages. A poly alpha-1,6-glucan ether compound may include a backbone of glucose monomer units wherein greater than or equal to 90% of the glucose monomer units are linked via alpha-1,6-glycosidic linkages and at least 5% of the glucose monomer units have branches via alpha-1,2 linkages. A poly alpha-1,6-glucan ether compound may include a backbone of glucose monomer units wherein greater than or equal to 90% of the glucose monomer units are linked via alpha-1,6-glycosidic linkages and at least 5% of the glucose monomer units have branches via alpha-1,3 linkages. A poly alpha-1,6-glucan ether compound may include a backbone of glucose monomer units wherein greater than or equal to 90% of the glucose monomer units are linked via alpha-1,6-glycosidic linkages and from about 5% to about 50% of the glucose monomer units have branches via alpha-1,2- or alpha-1,3-glycosidic linkages. A poly alpha-1,6-glucan ether compound may include a backbone of glucose monomer units wherein greater than or equal to 90% of the glucose monomer units are linked via alpha-1,6-glycosidic linkages and from about 5% to about 35% of the glucose monomer units have branches via alpha-1,2- or alpha-1,3-glycosidic linkages.

The poly alpha-1,6-glucan ether compounds disclosed herein can have a weight average molecular weight of 1000 to 500,000 Da (e.g., 10,000 to 400,000 daltons, 40,000 to 300,000 daltons, 80,000 to 300,000 daltons, 100,000 to 250,000 daltons, 150,000 to 250,000 daltons 180,000 to 225,000 daltons, or even 180,000 to 200,000 daltons). In some instances, it may be desirable to tailor the polymer size based on the intended application and/or benefit. The poly alpha-1,6-glucan ether compounds disclosed herein can be derived from a poly alpha-1,6-glucan having a weight average molecular weight of 900 to 450,000 Da, determined prior to substitution with the least one positively charged organic group. The poly alpha-1,6-glucan ether compounds disclosed herein can be derived from a poly alpha-1,6-glucan having a weight average molecular weight of 5000 to 400,000 DaDa (e.g., 10,000 to 350,000 Da, 50,000 to 350,000 Da 90,000 to 300,000 Da, 125,000 to 250,000 Da, or even 150,000 to 200,000 Da). Differently sized feedstock or backbone polymers may be preferred for different applications, or depending on the intended degree of substitution.

It would be understood by those skilled in the art that, since a cationic poly alpha-1,6-glucan ether compound as disclosed herein can have a degree of substitution between about 0.001 to about 3.0, the substituents on the polysaccharide cannot only be hydroxyl. The degree of substitution of a poly alpha-1,6-glucan ether compound can be stated with reference to a specific substituent or with reference to the overall degree of substitution, that is, the sum of the DoS of each different substituent for an ether compound as defined herein. As used herein, when the degree of substitution is not stated with reference to a specific substituent or substituent type, the overall degree of substitution of the cationic poly alpha-1,6-glucan ether compound is meant. The degree of substitution may be a cationic degree of substitution, or even a net cationic degree of substitution. The target DoS can be chosen to provide the desired solubility and performance of a composition comprising a cationic poly alpha-1,6-glucan ether compound in the specific application of interest. Cationic poly alpha-1,6-glucan ether compounds disclosed herein may have a DoS with respect to a positively charged organic group in the range of about 0.001 to about 3. A cationic poly alpha-1,6-glucan ether may have a DoS of about 0.01 to about 1.5. The poly alpha-1,6-glucan ether may have a DoS of about 0.01 to about 0.7. The poly alpha-1,6-glucan ether may have a DoS of about 0.01 to about 0.4. The poly alpha-1,6-glucan ether may have a DoS of about 0.01 to about 0.2. The DoS of the poly alpha-1,6-glucan ether compound can be at least about 0.001, 0.005, 0.1, 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. The DoS may be from about 0.01 to about 1.5, preferably from about 0.01 to about 1.0, more preferably from about 0.01 to about 0.8, more preferably from about 0.03 to about 0.7, or from about 0.04 to about 0.6, or from about 0.05 to about 0.5. For performance reasons in through-the-wash applications (e.g., a laundry or manual dishwashing detergent used in a wash cycle), it may be preferable for the DoS to be from about 0.01 to about 0.5, or from about 0.01 to about 0.25, or from about 0.01 to about 0.2, or from about 0.03 to about 0.15, or from about 0.04 to about 0.12. For performance reasons in through-the-rinse applications (e.g., a liquid fabric enhancer used in a rinse cycle), it may be preferably for the DoS to be from about 0.01 to about 1, or from about 0.03 to about 0.8, or from about 0.04 to about 0.7, or from about 0.05 to about 0.6, or from about 0.2 to about 0.8, or from about 0.2 to about 0.6, or from about 0.3 to about 0.6, or from about 0.4 to about 0.6. The DoS of the poly alpha-1,6-glucan may be from 0.01 to about 0.6, more preferably from 0.02 to about 0.5.

The cationic poly alpha-1,6-glucan ether compounds may be characterized by a cationic charge density. Cationic charge density may be expressed as milliequivalents of charge per gram of compound (meq/mol) and may be determined according to the method described in more detail below. It can be important to tailor the charge density of the cationic poly alpha-1,6-glucan ether compounds to provide the desired salt tolerance needed for the final composition. If the charge density is too low.

The cationic poly alpha-1,6-glucan ether compounds of the present disclosure may be characterized by a cationic charge density (or “CCD”) of from about 0.05 to about 12 meq/g, or from about 0.1 to about 8 meq/g, or from about 0.1 to about 4 meq/g, or from about 0.1 to about 3 meq/g, or from about 0.1 to about 2.6 meq/g.

A positively charged organic group includes a chain of one or more carbons having one or more hydrogens substituted with another atom or functional group, wherein one or more of the substitutions is with a positively charged group. The term “chain” as used herein encompasses linear, branched, and cyclic arrangements of carbon atoms, as well as combinations thereof.

The poly alpha-1,6-glucan derivative includes poly alpha-1,6-glucan substituted with at least one positively charged organic group on the polysaccharide backbone and/or on one or more of the optional branches. When substitution occurs on a glucose monomer contained in the backbone, the polysaccharide is derivatized at the 2, 3, and/or 4 glucose carbon position(s) with an organic group as defined herein which is linked to the polysaccharide through an ether (—O—) linkage in place of the hydroxyl group originally present in the underivatized (unsubstituted) poly alpha-1,6-glucan. When substitution occurs on a glucose monomer contained in a branch, the polysaccharide is derivatized at the 1, 2, 3, 4, or 6 glucose carbon position(s) with a positively charged organic group as defined herein which is linked to the polysaccharide through an ether (—O—) linkage.

A poly alpha-1,6-glucan ether compound as disclosed herein is termed a glucan “ether” herein by virtue of comprising the substructure —CG—O—CR—, wherein “—CG—” represents a carbon of a glucose monomer unit of a poly alpha-1,6-glucan ether compound, and wherein “—CR—” is included in the positively charged organic group. A cationic poly alpha-1,6-glucan monoether contains one type of a positively charged organic group. A cationic poly alpha-1,6-glucan mixed ether contains two or more types of positively charged organic groups. Mixtures of cationic poly alpha-1,6-glucan ether compounds can also be used.

Treatment compositions disclosed herein can comprise, or consist essentially of, one or more cationic poly alpha-1,6-glucan ether compounds as disclosed herein. A treatment composition may comprise one poly alpha-1,6-glucan ether compound. A treatment composition may comprise two or more poly alpha-1,6-glucan ether compounds, for example wherein the positively charged organic groups are different.

A treatment composition may comprise one or more cationic poly alpha-1,6-glucan ether compounds as disclosed herein, and may further comprise unsubstituted and/or non-cationic poly alpha-1,6-glucan compounds, which may be residual reactants that are unreacted/unsubstituted, or may have hydrolyzed. Typically, a low level of unsubstituted/non-cationic poly alpha-1,6-glucan compounds is preferred, as low levels may be indicative of reaction completeness with regard to the substitution, and/or chemical stability of the compounds in the treatment composition. The weight ratio of the cationic poly alpha-1,6-glucan ether compounds to unsubstituted/non-cationic poly alpha 1,6-glucan compounds may be 95:5 or greater, preferably 98:2 or greater, more preferably 99:1 or greater.

A “positively charged organic group” as used herein refers to a chain of one or more carbons that has one or more hydrogens substituted with another atom or functional group, wherein one or more of the substitutions is with a positively charged group. A positively charged group is typically bonded to the terminal carbon atom of the carbon chain. A positively charged organic group is considered to have a net positive charge since it comprises one or more positively charged groups, and comprises a cation (a positively charged ion). An organic group or compound that is “positively charged” typically has more protons than electrons and is repelled from other positively charged substances, but attracted to negatively charged substances. An example of a positively charged groups includes a substituted ammonium group. A positively charged organic group may have a further substitution, for example with one or more hydroxyl groups, oxygen atoms (forming a ketone group), alkyl groups, and/or at least one additional positively charged group.

A positively charged organic group may comprise a substituted ammonium group, which can be represented by Structure II:

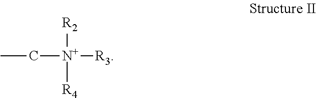

In Structure II, R₂, R₃ and R₄ may each independently represent a hydrogen atom, an alkyl group, or a C₆-C₂₄ aryl group. The carbon atom (C) shown in Structure II is part of the carbon chain of the positively charged organic group. The carbon atom is either directly ether-linked to a glucose monomer of poly alpha-1,6-glucan, or is part of a chain of two or more carbon atoms ether-linked to a glucose monomer of poly alpha-1,6-glucan. The carbon atom shown in Structure II can be —CH₂—, —CH—(where a H is substituted with another group such as a hydroxy group), or —C— (where both H's are substituted).

When R₂, R₃ and/or R₄ represent an alkyl group, the alkyl group can be a C₁-C₃₀ alkyl group, for example a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, C₂₅, C₂₆, C₂₇, C₂₈, C₂₉, or C₃₀ group. The alkyl group can be a C₁-C₂₄ alkyl group, or a C₁-C₁₈ or a C₆-C₂₀ alkyl group, or a C₁₀-C₁₆ alkyl group, or a C₁-C₄ alkyl group. When a positively charged organic group comprises a substituted ammonium group which has two or more alkyl groups, each alkyl group can be the same as or different from the other.

When R₂, R₃ and/or R₄ represent an aryl group, the aryl group can be a C₆-C₂₄ aryl group, optionally substituted with alkyl substituents. The aryl group can be a C₁₂-C₂₄ aryl group, optionally substituted with alkyl substituents, or a C₆-C₁₈ aryl group, optionally substituted with alkyl substituents.

A substituted ammonium group can be a “primary ammonium group”, “secondary ammonium group”, “tertiary ammonium group”, or “quaternary ammonium” group, depending on the composition of R₂, R₃ and R₄ in Structure II. A primary ammonium group is an ammonium group represented by Structure II in which each of R₂, R₃ and R₄ is a hydrogen atom (i.e., —C—NH3+).

A secondary ammonium group is an ammonium group represented by Structure II in which each of R₂ and R₃ is a hydrogen atom and R₄ is a C₁-C₃₀ alkyl group or a C₆-C₂₄ aryl group. A “secondary ammonium poly alpha-1,6-glucan ether compound” comprises a positively charged organic group having a monoalkylammonium group. A secondary ammonium poly alpha-1,6-glucan ether compound can be represented in shorthand as a monoalkylammonium poly alpha-1,6-glucan ether, for example monomethyl-, monoethyl-, monopropyl-, monobutyl-, monopentyl-, monohexyl-, monoheptyl-, monooctyl-, monononyl-, monodecyl-, monoundecyl-, monododecyl-, monotridecyl-, monotetradecyl-, monopentadecyl-, monohexadecyl-, monoheptadecyl-, or monooctadecyl-ammonium poly alpha-1,6-glucan ether. These poly alpha-1,6-glucan ether compounds can also be referred to as methyl-, ethyl-, propyl-, butyl-, pentyl-, hexyl-, heptyl-, octyl-, nonyl-, decyl-, undecyl-, dodecyl-, tridecyl-, tetradecyl-, pentadecyl-, hexadecyl-, heptadecyl-, or octadecyl-ammonium poly alpha-1,6-glucan ether compounds, respectively. An octadecyl ammonium group is an example of a monoalkylammonium group wherein each of R₂ and R₃ is a hydrogen atom and R₄ is an octadecyl group. It would be understood that a second member (i.e., R₁) implied by “secondary” in the above nomenclature is the chain of one or more carbons of the positively charged organic group that is ether-linked to a glucose monomer of poly alpha-1,6-glucan.

A tertiary ammonium group is an ammonium group represented by Structure II in which R₂ is a hydrogen atom and each of R₃ and R₄ is independently a C₁-C₂₄ alkyl group or a C₆-C₂₄ aryl group. The alkyl groups can be the same or different. A “tertiary ammonium poly alpha-1,6-glucan ether compound” comprises a positively charged organic group having a dialkylammonium group. A tertiary ammonium poly alpha-1,6-glucan ether compound can be represented in shorthand as a dialkylammonium poly alpha-1,6-glucan ether, for example dimethyl-, diethyl-, dipropyl-, dibutyl-, dipentyl-, dihexyl-, diheptyl-, dioctyl-, dinonyl-, didecyl-, diundecyl-, didodecyl-, ditridecyl-, ditetradecyl-, dipentadecyl-, dihexadecyl-, diheptadecyl-, or dioctadecyl-ammonium poly alpha-1,6-glucan ether. A didodecyl ammonium group is an example of a dialkyl ammonium group, wherein R₂ is a hydrogen atom and each of R₃ and R₄ is a dodecyl group. It would be understood that a third member (i.e., R₁) implied by “tertiary” in the above nomenclature is the chain of one or more carbons of the positively charged organic group that is ether-linked to a glucose monomer of poly alpha-1,6-glucan.

A quaternary ammonium group is an ammonium group represented by Structure II in which each of R₂, R₃ and R₄ is independently a C₁-C₃₀ alkyl group or a C₆-C₂₄ aryl group (i.e., none of R₂, R₃ and R₄ is a hydrogen atom).

A quaternary ammonium poly alpha-1,6-glucan ether compound may comprise a trialkyl ammonium group, where each of R₂, R₃ and R₄ is independently a C₁-C₃₀ alkyl group. The alkyl groups can all be the same, or two of the alkyl groups can be the same and one different from the others, or all three alkyl groups can be different from one another. A quaternary ammonium poly alpha-1,6-glucan ether compound can be represented in shorthand as a trialkylammonium poly alpha-1,6-glucan ether, for example trimethyl-, triethyl-, tripropyl-, tributyl-, tripentyl-, trihexyl-, triheptyl-, trioctyl-, trinonyl-, tridecyl-, triundecyl-, tridodecyl-, tritridecyl-, tritetradecyl-, tripentadecyl-, trihexadecyl-, triheptadecyl-, or trioctadecyl-ammonium poly alpha-1,6-glucan ether. It would be understood that a fourth member (i.e., R₁) implied by “quaternary” in this nomenclature is the chain of one or more carbons of the positively charged organic group that is ether-linked to a glucose monomer of poly alpha-1,6-glucan. A trimethylammonium group is an example of a trialkyl ammonium group, wherein each of R₂, R₃ and R₄ is a methyl group.

A positively charged organic group comprising a substituted ammonium group represented by Structure II can have each of R₂, R₃ and R₄ independently represent a hydrogen atom or an aryl group, such as a phenyl or naphthyl group, or an aralkyl group such as a benzyl group, or a cycloalkyl group such as cyclohexyl or cyclopentyl. Each of R₂, R₃ and R₄ may further comprise an amino group or a hydroxyl group.

The substituted ammonium group of the positively charged organic group is a substituent on a chain of one or more carbons that is ether-linked to a glucose monomer of the alpha-1,6-glucan. The carbon chain may contain from one to 30 carbon atoms. The carbon chain may be linear. Examples of linear carbon chains include, for example, —CH₂—, —CH₂CH₂—, —CH₂CH₂CH₂—, —CH₂(CH₂)2CH₂—, —CH₂(CH₂)3CH₂—, —CH₂(CH₂)4CH₂—, —CH₂(CH₂)5CH₂—, —CH₂(CH₂)6CH₂13 , —CH₂(CH₂)7CH₂—, —CH₂(CH₂)8CH₂—, —CH₂(CH₂)9CH₂—, and —CH₂(CH₂)10CH₂—; longer carbon chains can also be used, if desired. The carbon chain may be branched, meaning the carbon chain is substituted with one or more alkyl groups, for example methyl, ethyl, propyl, or butyl groups. The point of substitution can be anywhere along the carbon chain. Examples of branched carbon chains include —CH(CH₃)CH₂—, —CH(CH₃)CH₂CH₂—, —CH₂CH(CH₃)CH₂—, —CH(CH₂CH₃)CH₂—, —CH(CH₂CH₃)CH₂CH₂—, —CH₂CH(CH₂CH₃)CH₂—, —CH(CH₂CH₂CH₃)CH₂—, —CH(CH₂CH₂CH₃)CH₂CH₂—, and —CH₂CH(CH₂CH₂CH₃)CH₂—; longer branched carbon chains can also be used, if desired. Where the positively charged group is a substituted ammonium group, the first carbon atom in the chain is ether-linked to a glucose monomer of the poly alpha-1,6-glucan, and the last carbon atom of the chain in each of these examples is represented by the C in Structure II.

The chain of one or more carbons may be further substituted with one or more hydroxyl groups. Examples of a carbon chain having one or more substitutions with a hydroxyl group include hydroxyalkyl (e.g., hydroxyethyl, hydroxypropyl, hydroxybutyl, hydroxypentyl, hydroxyhexyl, hydroxyheptyl, hydroxyoctyl) groups and dihydroxyalkyl (e.g., dihydroxyethyl, dihydroxypentyl, dihydroxyhexyl, dihydroxyheptyl, dihydroxypropyl, dihydroxybutyl, dihydroxyoctyl) groups. Examples of hydroxyalkyl and dihydroxyalkyl (diol) carbon chains include —CH(OH)—, —CH(OH)CH₂—, —C(OH)₂CH₂—, —CH₂CH(OH)CH₂—, —CH(OH)CH₂CH₂—, —CH(OH)CH(OH)CH₂—, —CH₂CH₂CH(OH)CH₂—, —CH₂CH(OH)CH₂CH₂—, —CH(OH)CH₂CH₂CH₂—, —CH₂CH(OH)CH(OH)CH₂—, —CH(OH)CH(OH)CH₂CH₂— and —CH(OH)CH₂CH(OH)CH₂—. In each of these examples, the first carbon atom of the chain is ether-linked to a glucose monomer of poly alpha-1,6-glucan, and the last carbon atom of the chain is linked to a positively charged group. Where the positively charged group is a substituted ammonium group, the last carbon atom of the chain in each of these examples is represented by the C in Structure II.

An example of a quaternary ammonium poly alpha-1,6-glucan ether compound is trimethylammonium hydroxypropyl poly alpha-1,6-glucan. The positively charged organic group of this ether compound can be represented by the following structure:

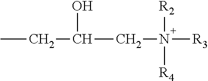

where each of R₂, R₃ and R₄ is a methyl group. The structure above is an example of a quaternary ammonium hydroxypropyl group.

Where a carbon chain of a positively charged organic group has a substitution in addition to a substitution with a positively charged group, such additional substitution may be with one or more hydroxyl groups, oxygen atoms (thereby forming an aldehyde or ketone group), alkyl groups (e.g., methyl, ethyl, propyl, butyl), and/or additional positively charged groups. A positively charged group is typically bonded to the terminal carbon atom of the carbon chain. A positively charged group can also comprise one or more imidazoline rings.

A cationic poly alpha-1,6-glucan ether compound as disclosed herein may be a salt. The counter ion for the positively charged organic group can be any suitable anion, including an acetate, borate, bromate, bromide, carbonate, chlorate, chloride, chlorite, dihydrogen phosphate, fluoride, hydrogen carbonate, hydrogen phosphate, hydrogen sulfate, hydrogen sulfide, hydrogen sulfite, hydroxide, hypochlorite, iodate, iodide, nitrate, nitride, nitrite, oxalate, oxide, perchlorate, permanganate, phosphate, phosphide, phosphite, silicate, stannate, stannite, sulfate, sulfide, sulfite, tartrate, or thiocyanate anion, preferably chloride. In an aqueous solution, a poly alpha-1,6-glucan ether compound is in a cationic form. The positively charged organic groups of a cationic poly alpha-1,6-glucan ether compound can interact with salt anions that may be present in an aqueous solution.

The poly alpha-1,6-glucan ether compound may comprise a positively charged organic group, wherein the positively charged organic group comprises a substituted ammonium group. From about 0.5% to about 50% of the backbone glucose monomer units of the ether compound may have branches via alpha-1,2 glycosidic linkages, and the positively charged organic group may comprise a substituted ammonium group. From about 5% to about 30% of the backbone glucose monomer units of the ether compound may have branches via alpha-1,2 glycosidic linkages, and the substituted ammonium group may comprise a substituted ammonium group. From about 0.5% to about 50% of the backbone glucose monomer units of the ether compound may have branches via alpha-1,2 glycosidic linkages, and the substituted ammonium group may comprise a trimethyl ammonium group. From about 5% to about 35% of the backbone glucose monomer units of the ether compound may have branches via alpha-1,2 glycosidic linkages, and the substituted ammonium group may comprise a trimethyl ammonium group.

The poly alpha-1,6-glucan ether compound may comprise a positively charged organic group, wherein the positively charged organic group comprises a trimethylammonium hydroxyalkyl group. From about 0.5% to about 50% of the backbone glucose monomer units of the ether compound may have branches via alpha-1,2 glycosidic linkages, and the positively charged organic group may comprise a trimethylammonium hydroxyalkyl group. From about 5% to about 30% of the backbone glucose monomer units of the ether compound may have branches via alpha-1,2 glycosidic linkages, and the positively charged organic group may comprise a trimethylammonium hydroxyalkyl group. From about 0.5% to about 50% of the backbone glucose monomer units of the ether compound may have branches via alpha-1,2 glycosidic linkages, and the trimethylammonium hydroxyalkyl group may comprise a trimethylammonium hydroxypropyl group. From about 5% to about 30% of the backbone glucose monomer units of the ether compound may have branches via alpha-1,2 glycosidic linkages, and the trimethylammonium hydroxyalkyl group may comprise a trimethylammonium hydroxypropyl group.

The poly alpha-1,6-glucan ether compound may comprise a positively charged organic group, wherein the positively charged organic group comprises a substituted ammonium group comprising a quaternary ammonium group. From about 0.5% to about 50% of the backbone glucose monomer units of the ether compound may have branches via alpha-1,2 glycosidic linkages, and the quaternary ammonium group may comprise at least one C1 to C18 alkyl group. From about 5% to about 30% of the backbone glucose monomer units of the ether compound may have branches via alpha-1,2 glycosidic linkages, the quaternary ammonium group may comprise at least one C1 to C18 alkyl group. From about 0.5% to about 50% of the backbone glucose monomer units of the ether compound may have branches via alpha-1,2 glycosidic linkages, and the quaternary ammonium group may comprise at least one C1 to C4 alkyl group. From about 5% to about 30% of the backbone glucose monomer units of the ether compound may have branches via alpha-1,2 glycosidic linkages, and the quaternary ammonium group may comprise at least one C1 to C4 alkyl group. From about 0.5% to about 50% of the backbone glucose monomer units of the ether compound may have branches via alpha-1,2 glycosidic linkages, and the quaternary ammonium group may comprise at least one C10 to C16 alkyl group. From about 5% to about 30% of the backbone glucose monomer units of the ether compound may have branches via alpha-1,2 glycosidic linkages, and the quaternary ammonium group may comprise at least one C10 to C16 alkyl group.

The poly alpha-1,6-glucan ether compound may comprise a quaternary ammonium group comprising one C10 to C16 alkyl group, where the quaternary ammonium group further comprises two methyl groups. From about 0.5% to about 50% of the backbone glucose monomer units of the ether compound may have branches via alpha-1,2 glycosidic linkages, and the quaternary ammonium group may comprise one C10 to C16 alkyl group further comprises two methyl groups. From about 5% to about 30% of the backbone glucose monomer units of the ether compound may have branches via alpha-1,2 glycosidic linkages, and the quaternary ammonium group may comprise one C10 to C16 alkyl group further comprises two methyl groups.

From about 0.5% to about 50% of the backbone glucose monomer units of the ether compound may have branches via alpha-1,2 glycosidic linkages, and the quaternary ammonium group may comprise one C10 alkyl group and two methyl groups. From about 5% to about 30% of the backbone glucose monomer units of the ether compound may have branches via alpha-1,2 glycosidic linkages, and the quaternary ammonium group may comprise one C10 alkyl group and two methyl groups.

The cationic poly alpha-1,6-glucan ether compound may comprise a positively charged organic group, wherein the positively charged organic group comprises a quaternary ammonium hydroxyalkyl group. From about 0.5% to about 50% of the backbone glucose monomer units of the ether compound may have branches via alpha-1,2 glycosidic linkages, and the positively charged organic group may comprise a quaternary ammonium hydroxyalkyl group. From about 5% to about 30% of the backbone glucose monomer units of the ether compound may have branches via alpha-1,2 glycosidic linkages, and the positively charged organic group may comprise a quaternary ammonium hydroxyalkyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxymethyl group, a quaternary ammonium hydroxyethyl group, or a quaternary ammonium hydroxypropyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxymethyl group, a quaternary ammonium hydroxyethyl group, or a quaternary ammonium hydroxypropyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxymethyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxymethyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxyethyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxyethyl group. From about 0.5% to about 50% of the backbone glucose monomer units of the ether compound may have branches via alpha-1,2 glycosidic linkages, and the quaternary ammonium hydroxyalkyl group may comprise a quaternary ammonium hydroxypropyl group. From about 5% to about 30% of the backbone glucose monomer units of the ether compound may have branches via alpha-1,2 glycosidic linkages, and the quaternary ammonium hydroxyalkyl group may comprise a quaternary ammonium hydroxypropyl group.

Examples of cationically modified poly alpha-1,6-glucan ether compounds and methods of making them are disclosed in U.S. Pat. No. 11,905,495 and US 20230045856.

### Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 10%, 7%, 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Additional Ingredients

The personal care compositions described herein may include a variety of optional ingredients to tailor the properties and characteristics of the composition, as desired. The optional ingredients may be materials that are commonly included in compositions of the type. The optional ingredients should be physically and chemically compatible with the essential components of the personal care composition and should not otherwise unduly impair the stability, aesthetics, or performance of the composition. Individual concentrations of optional components can generally range from 0.001% to 10%.

Some non-limiting examples of optional ingredients that can be included in the personal care compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Cationic Polymer

The personal care compositions herein may include 0.05-3% of a cationic polymer (e.g., 0.1-2%, or even 0.2-0.8%) besides the cationic poly alpha-1,6-glucan ether compound to provide improved appearance, feel or deposition benefits to hair or skin. The cationic polymer can have a weight average molecular weight of 50 kDa to about 5 MDa (e.g., 500 kDa-4 MDa, 1-3 MDa, 1.2-2 MDa, or even 1.4-1.8 MDa) and a charge density of 0.2 meq/g to 12 meq/g (e.g., 0.4-10 meq/g, 0.4-5 meq/g, 0.4-4 meq/g, 0.4-3 meq/g, or even 0.4-2 meq/g). The charge densities can be measured at the pH of intended use of the personal care composition, which can be pH 3 to pH 9 (e.g., pH 4-8 or pH 4.5-6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some nonlimiting examples of cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some nonlimiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Personal Care Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl- , trichloride (Polyquaternium-76). AM:TRIQUAT may have a charge density of 1.6 meq/g and a molecular weight of 1.1 MDa.

In some aspects, the cationic monomer can be polymethyacrylamidopropyl trimonium chloride, available under the trade name Polycare® 133, from Solvay (Brussels, Belgium). Copolymers of the cationic monomer may also suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™M JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Pat. No. 6,930,078.

In some instances, the cationic polymer may include a synthetic cationic polymer or derivative thereof present at 0.025% to about 5%. Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

### Gel Network

The personal care composition may also include a fatty alcohol gel network. Gel networks are formed by combining fatty alcohols and surfactants in the ratio ranging from 1:1 to 40:1 (e.g., 2:1 to 20:1 or 3:1 to 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to personal care compositions, such as stabilizing and conditioning benefits.

A fatty alcohol can be included in the gel network at a level by weight of from about 0.05%, by weight, to about 14%, by weight. For example, the fatty alcohol can be included in an amount ranging from about 1%, by weight, to about 10%, by weight,, and/or from about 6%, by weight, to about 8%, by weight.

Suitable fatty alcohols include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

A gel network can be prepared by charging a vessel with water. The water can then be heated to about 74° C. Cetyl alcohol, stearyl alcohol, and surfactant can then be added to the heated water. After incorporation, the resulting mixture can passed through a heat exchanger where the mixture is cooled to about 35° C. Upon cooling, the fatty alcohols and surfactant crystallized can form crystalline gel network. Table 1 provides the components and their respective amounts for an example gel network composition.

To prepare the gel network pre-mix of Table A, water is heated to about 74° C. and the fatty alcohol and gel network surfactant are added to it in the quantities depicted in Table A. After incorporation, this mixture is passed through a mill and heat exchanger where it is cooled to about 32° C. As a result of this cooling step, the fatty alcohol, the gel network surfactant, and the water form a crystalline gel network.

TABLE A

| Premix | % |
|---|---|
| Gel Network Surfactant¹ | 11.00 |
| Stearyl Alcohol | 8% |
| Cetyl Alcohol | 4% |
| Water | QS |

For anionic gel networks, suitable gel network surfactants above include surfactants with a net negative charge including sulfonates, carboxylates and phosphates among others and mixtures thereof. For cationic gel networks, suitable gel network surfactants above include surfactants with a net positive charge including quaternary ammonium surfactants and mixtures thereof. For Amphoteric or Zwitterionic gel networks, suitable gel network surfactants above include surfactants with both a positive and negative charge at product usage pH including betaines, amine oxides, sultaines, amino acids among others and mixtures thereof.

### Method of Making a Personal Care Composition

The personal care composition described herein can be made using conventional methods for making compositions of the type desired (e.g., shampoo, conditioner or body wash). A particularly suitable method of making the compositions herein is described in Example 1 below. In some aspects, the composition may include a gel network to aid in the conditioning of hair or scalp. U.S. Publication No. 2006/269501 discloses methods of making gel networks that may be suitable for use herein.

### Method of Use

The personal care compositions described herein can be used in a conventional manner for cleansing and conditioning of hair or skin. Effective amounts of the composition for use generally range from 1 g to 50 g (e.g., 1 g to about 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid, solid, semi-solid, flake, gel, foam, in a pressurized container with a propellant added, or used in a pump spray form. The viscosity of the product may be selected to accommodate the form desired.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and/or conditioning benefit.

### Methods

### Rheology

Personal care composition viscosities can be measured on a 2.5 mL sample using a cone and plate Brookfield® RS brand rheometer or equivalent with cone C75-1, shear rate of 2 s⁻¹, temperature of 27° C. and time of 3 minutes.

### Percent Transmittance (% T)

This method can be used to determine the transparency or opacity of a composition by measuring the transmission of UV/VIS light through a sample of the composition with a spectrophotometer. A light wavelength of 600 nm has been shown to be adequate for characterizing % T. Before testing, the spectrophotometer should be calibrated by measuring a “blank” and calibrating the readout to 100% transmittance. The spectrophotometer is not particularly limited as long as it can measure transmittance at 600 nm. For example, a SpectraMax® M-5 brand spectrophotometer with Software Pro® v.5TM software available from Molecular Devices may be suitable. It is important to ensure that no air bubbles are present in the sample prior to measuring. When using % T to determine whether an in situ coacervate is present, it may be desirable to use a clear test sample. For example, some compositions that contain silicone or certain fatty alcohols may be cloudy or hazy, and thus have a lower % T, which may be interpreted as a false positive for the presence of an in situ coacervate. Removing these ingredients should not affect the presence or absence of an in situ coacervate.

### Hair Treatment & Hair Wet Feel Friction Measurement (Initial and Final Rinse Friction)

This method uses a hair substrate (hair switch) to measure the conditioning properties of a composition or material. The hair switch is in the form of a round ponytail made of hair tresses of the general population tested. The hair switch is 20.3 cm long and weight 4 grams. Suitable hair switches for conducting the tests below can be obtained from International Hair Importers & Products Inc. in Glendale, NY.

Water temperature is set at 100° F., hardness is 7 grain per gallon, and flow rate is 1.6 liter per minute. For shampoos in liquid form, 0.2 ml of a liquid shampoo is applied on the hair switch in a zigzag pattern uniformly to cover the entire hair length, using a syringe. For shampoo in aerosol foam form, foam shampoo is dispensed to a weighing pan on a balance. 0.2 grams of foam shampoo is taken out from weighing pan and applied on the hair switch uniformly to cover the entire hair length via a spatula. The hair switch is then 1st lathered for 30 seconds, rinse with water for 30 seconds, and 2nd lathered for 30 seconds (0.05 ml of shampoo per gram of hair per lather, for 2 lathers; this totals 0.1 ml of shampoo per gram of hair). Water flow rate is then reduced to 0.2 liter per minute. The hair switch is sandwiched with a clamp under 1800 gram of force and pulled through the entire length while the water is running at the low flow rate. The pull time is 30 second. Friction is measured with a friction analyzer with a load cell of 5 kg. Repeat the pull under rinse for total of 21 times. Total 21 friction values are collected. The final rinse friction is the average friction of the last 7 points and initial rinse friction is the average of the initial 7 points. The delta final to initial is calculated by subtracting the final rinse friction from the initial rinse friction.

### Combing Force

The force required to comb a hair switch after treatment (wet and dry) was measured using the Texture Analyzer TA-XT Plus (manufactured by Stable Micro Systems), Instron 5542 (manufactured by Instron) or equivalent force measurement device. This method is an industry standard method for measuring wet/dry hair combing forces, disclosed by TRI Princeton.

The hair switch is placed within the holder of the Texture Analyzer, fixed at the root end of the hair switch. The hair switch is positioned within the combs in series and then pulled through the combs by the Texture Analyzer, while the average force to pull the switch through each comb is recorded (=1 combing stroke). The hair switch is disengaged from the combs and returned to its pre-combing position. The hair switch is then combed 9 more times using the same combing procedure. The outputs include work (gram force, gf) to detangle and combing at tips (resistance at tips).

### Wet Force to Comb

### Dry Force to Comb

### Dry Smoothness

### Dry—Feeling Moisturized

### EXAMPLES

The examples provided below are intended to be illustrative in nature and are not intended to be limiting.

### Example 1: Formulations

Table 1 provides examples of inventive personal care composition formulations.

TABLE 1

| | Control | Inv 1 | Inv 2 | Inv 3 | Inv 4 | Inv 5 | Inv 6 | Inv 7 | Inv 8 |
|---|---|---|---|---|---|---|---|---|---|
| Ingredient | wt % | | | | | | | | |
| SLS ¹ | 3.0 | 3.0 | | | | | | | |
| AOS ² | 7.0 | 7.0 | 5.0 | 7.0 | 7.0 | 7.0 | 7.0 | 7.0 | 7.0 |
| Decyl glucoside³ | | | | 3.0 | 3.0 | 3.0 | 3.0 | 3.0 | 3.0 |
| SLT ⁴ | | | 5.0 | | | | | | |
| CAPB ⁵ | 5.0 | 5.0 | 2.0 | 5.0 | 5.0 | 5.0 | 5.0 | 5.0 | 5.0 |
| Guar | 0.15 | 0.25 | 0.25 | 0.25 | — | 0.25 | — | — | — |
| hydroxypropyltrimonium | | | | | | | | | |
| chloride ⁶ | | | | | | | | | |
| Cationic poly alpha-1,6- | 0.00 | 0.75 | 0.75 | 0.75 | 0.75 | — | 0.50 | 0.75 | 1.00 |
| glucan ether ⁷ | | | | | | | | | |
| DC1872 ⁸ | 0.00 | 0.50 | — | — | — | — | — | — | — |
| Preservatives | 0.50 | 0.50 | 0.50 | .50 | .50 | .50 | .50 | .50 | .50 |
| Disodium EDTA | 0.16 | 0.16 | 0.16 | 0.16 | 0.16 | 0.16 | 0.16 | 0.16 | 0.16 |
| Sodium salicylate | 0.25 | 0.25 | 0.25 | .25 | .25 | .25 | .25 | .25 | .25 |
| Citric acid | 0.32 | 0.34 | 0.39 | — | — | — | — | — | — |
| Fragrance | 0.40 | 0.80 | 0.40 | 0.8 | 0.8 | 0.8 | 0.8 | 0.8 | 0.8 |
| EGDS ⁹ | 0.00 | — | 12.5 | — | — | — | — | — | — |
| Glycerin | 1.00 | 1.00 | 1.00 | — | — | — | — | — | — |
| DI water | qs | qs | qs | qs | qs | qs | qs | qs | qs |
| pH | 5.53 | 5.53 | 5.20 | 5.17 | 5.06 | — | — | — | — |
| Viscosity (mPa-s) | 5,752 | 6,879 | 8,599 | 4780 | 4561 | — | — | — | — |

¹ Sodium lauryl sulfate available from P&G Chemicals.
² Bio-Terge ® AS-40 HP (C14-16 alpha olefin sulfonate) available from Stepan.
³Plantaren ® 2000N UP available from Sino Lion
⁴ Sodium lauroyl taurate from P&G chemicals
⁵ Tego Betain ® available from Evonik
⁶ Jaguar Excel ® available from Solvay (MW = 1,100,000 Da, CD = .7)
⁷ Alpha-glucan hydroxypropyltrimonium chloride from IFF (MW = 185 kDa, CD = 0.8, DoS = 0.15)
⁸ XIAMETER ® MEM-1872 Emulsion from Dow
⁹ EGIN ® G 1100 from Evonik

### Example 2: Conditioning Benefits

This example demonstrates the conditioning benefits of the present cationic poly alpha-1,6-glucan ether compounds. Example formulation Inv 1 and Inv 3 from Table 1 were tested against a current market conditioning shampoo (Patent® Daily Moisture Renewal from The Procter & Gamble Company) for various standard conditioning benefits. The results of the test are summarized in Table 2.

TABLE 2

| | | Inv | Inv | Pantene Daily |
|---|---|---|---|---|
| | Control | 1 | 2 | Moisture Renewal |
| Wet feel (gf) | 1286.29 | 1107.45 | 1110.42 | 1040.79 |
| Final rinse friction | 1423.71 | 1423.65 | 1445.73 | 1466.16 |
| (gf) | | | | |
| Wet detangling | 7.04 | 6.86 | 6.97 | 6.21 |
| (log (wet area)) | | | | |
| Wet combing | 5.87 | 5.58 | 5.74 | 5.09 |
| (log(gf)) | | | | |
| Dry detangling | 5.59 | 5.42 | 5.30 | 5.27 |
| (log(dry area)) | | | | |
| Dry combing | 4.36 | 4.08 | 4.05 | 4.1 |
| (log(gf)) | | | | |
| Resistance at tips | 5.32 | 5.12 | 5.12 | 4.9 |
| (log(gf)) | | | | |
| Smoothness (gf) | 1807.62 | 1847.61 | 1873.03 | 1691.45 |
| Force to comb (gf) | 6817.7 | 6700.68 | 6783.87 | 6307 |

As can be seen in Table 2, the inventive compositions provide good conditioning.

### Example 3: Silicone Deposition

This example demonstrates the surprising ability of the cationic poly alpha-1,6-glucan ether compounds to deposit silicone on hair. Silicone is popular hair conditioning agent due to its low surface energy, high gas permeability (breathability), and excellent thermal stability. As a result, silicone-based hair products can reduce interfiber friction, increase softness, and impart hydrophobicity (water repellency), along with easier detangling, smoothness, shine, frizz control, UV and color protection, repair of damage, and shorter drying times.

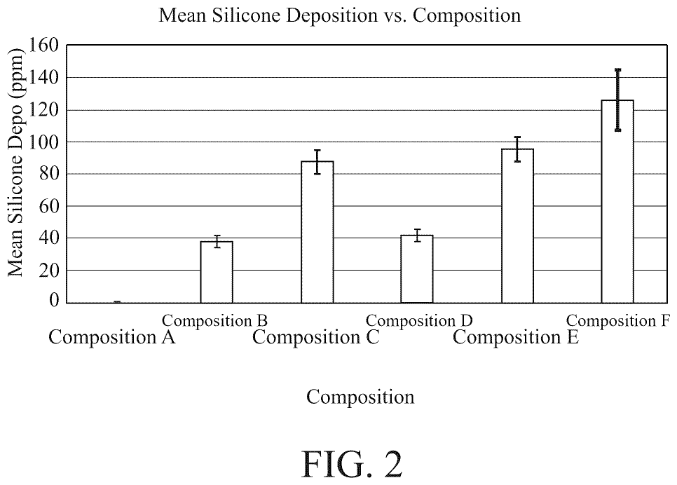

With traditional cationic polymers such as cellulose and guar, it has been previously demonstrated that silicone deposition typically increases with increased cationic polymer charge density. Surprisingly, the relatively low molecular weight cationic poly alpha-1,6-glucan ether compounds deposited more silicone to hair than guar hydroxypropyltrimonium chloride (NHance® 3196 from Ashland) and polyquaternium-10 (a cationic cellulose sold under the name UCARE® polymer LR30M by Dow), which both have higher estimated charge density. Surprisingly, even composition E with polyglucan DCPQ44 with at even lower charge density deposited more silicone than both comparative compositions. The results of the testing are summarized in Table 3 and illustrated in FIG. 2.

TABLE 3

| | Wt % | | | | | |
|---|---|---|---|---|---|---|
| Ingredient | A | B | C | D | E | F |
| Water | QS | QS | QS | QS | QS | QS |
| Lauramidopropyl betaine ¹ | 9.75 | 9.75 | 9.75 | 9.75 | 9.75 | 9.75 |
| Sodium cocoyl isethionate ² | 6.00 | 6.00 | 6.00 | 6.00 | 6.00 | 6.00 |
| Sodium laroyl sarcosinate ³ | 4.00 | 4.00 | 4.00 | 4.00 | 4.00 | 4.00 |
| Dimethiconol ⁴ | 0.50 | 0.50 | 0.50 | 0.50 | 0.50 | 0.50 |
| Fragrance | 0.85 | 0.85 | 0.85 | 0.85 | 0.85 | 0.85 |
| Polyquaternium-10 ⁵ | | 0.60 | | | | |
| Guar hydroxypropyltrimonium | | | 0.60 | | | |
| chloride ⁶ | | | | | | |
| Guar hydroxypropyltrimonium | | | | 0.60 | | |
| chloride ⁷ | | | | | | |
| Cationic poly alpha-1,6-glucan | | | | | 0.60 | |
| ether compound ⁸ | | | | | | |
| Cationic poly alpha-1,6-glucan | | | | | | 0.60 |
| ether compound ⁹ | | | | | | |
| Preservative | 0.4 | 0.4 | 0.4 | 0.4 | 0.4 | 0.4 |
| Citric acid (to pH 5.5) | 0.25 | 0.25 | 0.25 | 0.25 | 0.25 | 0.25 |
| Sodium citrate, dihydrate | 0 | 0 | 0 | 0 | 0 | 0 |
| Estimated Polymer Charge | N/A | 0.70 | 0.70 | 0.40 | 0.40 | 0.60 |
| Density: | | | | | | |
| Silicone Depo (ppm): | 0 | 38 | 88 | 42 | 95 | 126 |

¹ Mackam ® DAB ULS from Solvay
² Jordapon ® Cl Prill from BASF
³ SP Crodasinic ® LS30/NP MBAL-LQ-(RB) from Croda
⁴ Xiameter ® MEM-1872 Emulsion from Dow
⁵ UCARE ® polymer LR30M from Dow
⁶ NHance ® 3196 from Ashland
⁷ NHance ® 3000 from Ashland; MW = 1 MDa, CD = 0.37, DoS = 0.07
⁸ Alpha-glucan hydroxypropyltrimonium chloride from IFF (MW = 185 kDa, CD = 0.4, DoS = 0.07)
⁹ Alpha-glucan hydroxypropyltrimonium chloride from IFF (MW = 185 kDa, CD = 0.6, DoS = 0.11)

### Example 4: Coacervate Formation

This example demonstrates the surprising lack of in situ coacervate formation in compositions containing a cationic poly alpha-1,6-glucan ether compound with low charge density and low molecular weight. A common challenge when formulating with cationic polymers that have a low charge density and low MW is stability. Cationic polymers with low charge density and/or low MW can destabilize the composition by forming an in situ coacervate, which results in the composition having a hazy or turbid appearance. Thus, measuring the % T of the neat product can indicate whether an in situ coacervate is present. On the other hand, when the composition is diluted with water (e.g., during its intended use), a coacervate is desired.

The inventive compositions from Table 1 were tested according to the % T method to determine whether a coacervate was present. A lower % T indicates less light transmission, which corresponds to a higher amount of coacervate formation. Conversely, a higher % T indicates more light transmission and less coacervate. The test results are summarized in Table 4.

TABLE 4

| | % T | % T (600nm) |
|---|---|---|
| Invention | Neat | at 9:1 dilution |
| 1 | — | 52% |
| 2 | — | — |
| 3 | 98% | 18% |
| 4 | 98% | 30% |
| 5 | 90% | 60% |
| 6 | 90% | 55% |
| 7 | 90% | 18% |
| 8 | 90% | 11% |

As can be seen in Table 4, the compositions start out transparent (stable), and then form a coacervate, or a polymer-rich phase, upon dilution.

### Example 5: Combination of Cationic Polymer for Improved or Synergistic Dilute Coacervate Formation

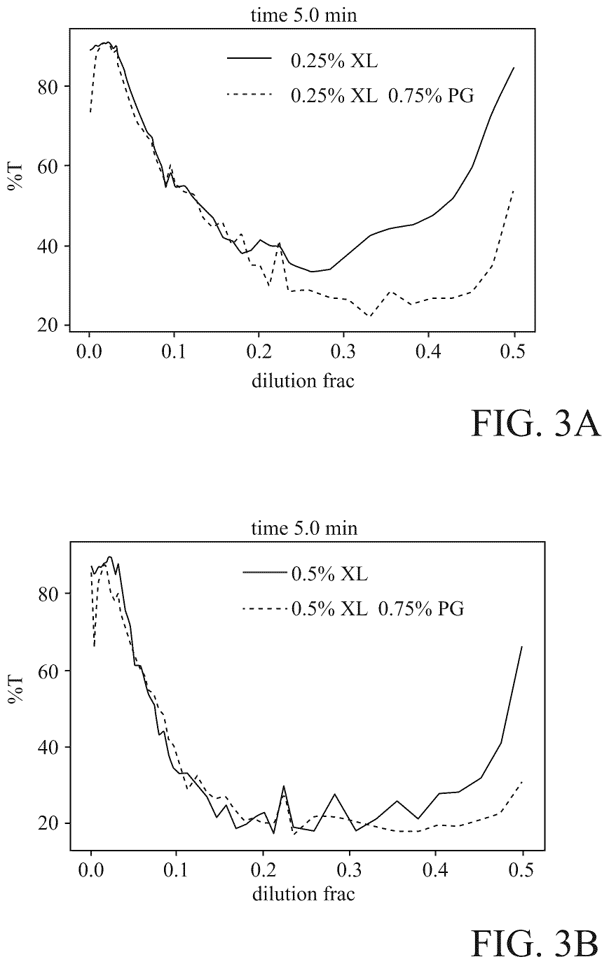

This example demonstrates the coacervate benefit obtained when a cationic poly alpha-1,6-glucan ether compound is combined with a guar hydroxypropyltrimonium chloride cationic polymer. In this example, compositions comprising only the guar cationic polymer were compared to compositions that contained the guar and the cationic poly alpha-1,6-glucan ether compound, as indicated in Table 5. % T at 600 nm was used to characterize the coacervate that formed five minutes after dilution. The results of the testing are summarized in Table 5 and illustrated in FIGS. 3A and 3B. As can be seen from the results, the combination of guar and polyglucan cationic polymers provided a better coacervate.

TABLE 5

| | | | |
|---|---|---|---|
| C1 | I1 | C2 | I2 |

Wt %

| | | | | |
|---|---|---|---|---|
| Sodium C14-16 alpha olefin | 5 | 5 | 5 | 5 |
| sulfonate ¹ | | | | |
| Sodium lauryl sulfate ² | 5 | 5 | 5 | 5 |
| Cocamidopropyl betaine ³ | 2 | 2 | 2 | 2 |
| Guar | 0.25 | 0.25 | 0.5 | .05 |
| hydroxypropyltrimonium | | | | |
| chloride ⁴ | | | | |
| poly alpha-1,6-glucan ether ⁵ | | 0.75 | | 0.75 |
| Water | Qs | Qs | Qs | Qs |

% T

| | | | | |
|---|---|---|---|---|
| at 1:1 dilution (0.5 dilution | 84.7 | 53.4 | 65.9 | 30.5 |
| fraction) | | | | |
| % T at 1:1.5 dilution (0.4% | 47.2 | 26.5 | 27.5 | 19.3 |
| dilution fraction) | | | | |
| % T at 1:2 dilution (0.33% | 42.3 | 21.8 | 20.8 | 18.9 |
| dilution fraction) | | | | |

¹ Bio-Terge ® AS-40 HP from Stepan.
² From P&G Chemicals
³ Tego ® Betain from Evonik
⁴ Jaguar Excel ® from Solvay
⁵ Alpha-glucan hydroxypropyltrimonium chloride from IFF (MW = 185 kDa, CD = 0.8, DoS = 0.15)

### Examples/Combinations

1. A personal care composition, comprising:
an anionic detersive surfactant;
an co-surfactant selected from non-ionic, amphoteric and zwitterionic surfactants;
a cationic polymer;
a cationically modified poly alpha-1,6-glucan ether compound; and
an aqueous carrier.

2. The personal care composition of paragraph 1, wherein the cationically modified poly alpha-1,6-glucan ether compound comprises a backbone of glucose monomer units, wherein at least 65% of the backbone glucose monomer units are linked via alpha-1,6-glycosidic linkages.

3. The personal care composition of paragraph 1 or 2, wherein the cationically modified poly alpha-1,6-glucan ether compound has a degree of substitution of 0.01 to 0.3.

4. The personal care composition of any one of paragraphs 1 to 3, wherein the cationically modified poly alpha-1,6-glucan ether compound has a weight average molecular weight of 50000 to 500,000 daltons.

5. The personal care composition of any one of paragraphs 1 to 4, wherein the cationically modified poly alpha-1,6-glucan ether compound has a charge density of 0.05 to 3.

6. The personal care composition of any one of paragraphs 1 to 5, wherein 5% to 35% of the backbone glucose monomer units have branches via alpha-1,2 and/or alpha-1,3-glycosidic linkages.

7. The personal care composition of any one of paragraphs 1 to 6, wherein the poly alpha-1,6-glucan ether compound comprises a cationically modified poly alpha-1,6-glucan substituted with at least one positively charged ammonium group.

8. The personal care composition of paragraph 7, wherein the substituted ammonium group comprises a quaternary ammonium group comprising at least one C10 to C16 alkyl group.

9. The personal care composition of paragraph 8, wherein the quaternary ammonium group comprises a trimethylammonium group.

10. The personal care composition of any one of paragraphs 1 to 9, wherein the cationically modified poly alpha-1,6-glucan ether compound improves at least one conditioning benefit selected from Wet Feel, Final Rinse Friction, Wet Detangling, Wet Combing, Dry Detangling, Dry Combing, Resistance at Tips, Smoothness and Force to Comb compared to a control composition that does not include the cationically modified poly alpha-1,6-glucan ether compound.

11. The personal care composition of paragraph 10, wherein the composition provides a better dilute coacervate than a control composition that does not include the cationically modified poly alpha-1,6-glucan ether compound, according to the % T method.

12. The personal care composition of any one of paragraphs 1 to 11, wherein the composition does not comprise an in-situ coacervate.

13. The personal care composition of paragraph 11, wherein the composition has a % T of greater than 80, preferably greater than 90, more preferably greater than 95.

14. The personal care composition of any one of paragraphs 1 to 13, wherein the composition reduces Frizz according to the Frizz test.

15. The personal care composition of any one of paragraphs 1 to 14, wherein the composition has a viscosity of 500 to 30,000 cP according to the Rheology Method.

16. The personal care composition of any one of paragraphs 1 to 15, wherein the anionic detersive surfactant is select from alkyl sulfates, alkyl ether sulfates, acyl glycinates, acyl sarcosinates, acyl glutamates, acyl alaninates, sulfosuccinates, isethionates, sulfonates, sulfoacetates, glucose carboxylates, alkyl ether carboxylates, acyl taurates, sodium, ammonium or potassium salts of these, and mixtures thereof.

17. The personal care composition of any one of paragraphs 1 to 16, wherein the co-surfactant is selected from aliphatic secondary and tertiary amines, wherein at least one aliphatic substituent contains 8 to 18 carbon atoms and at least one aliphatic substituent contains an anionic selected from carboxy, sulfonate, sulfate, phosphate, and phosphonate.

18. The personal care composition of paragraph 16, wherein the co-surfactant is selected from ocoamphoacetate, cocoamphodiacetate, lauroamphoacetate, lauroamphodiacetate, and mixtures thereof.

19. The personal care composition of any one of paragraphs 1 to 18, wherein the co-surfactant is selected from derivatives of aliphatic quaternary ammonium, phosphonium, and sulfonium compounds, wherein at least one aliphatic substituent contains 8 to 18 carbon atoms and at least one aliphatic substituent contains an anionic group selected from carboxy, sulfonate, sulfate, phosphate and phosphonate.

20. The personal care composition of paragraph 18, wherein the co-surfactant is selected from cocamidopropyl betaine, lauramidopropyl betaine and mixtures thereof.

21. The personal care composition of any one of paragraphs 1 to 20, further comprising a gel network.

22. The personal care composition of paragraphs 1 to 21, further comprising an additional ingredient.

23. A personal care composition, comprising:
an anionic detersive surfactant;
an amphoteric co-surfactant;
a cationically modified poly alpha-1,6-glucan ether compound; and
an aqueous carrier, wherein the composition is free of sulfated surfactants and exhibits improved stability compared to a control composition that does not contain the cationically modified poly alpha-1,6-glucan ether compound.

24. A personal care composition, comprising:
an anionic detersive surfactant;
an amphoteric co-surfactant;
a cationically modified poly alpha-1,6-glucan ether compound;
a silicone conditioning agent; and
an aqueous carrier, wherein the composition exhibits improved deposition of the silicone conditioning agent according to the Deposition test when compared to a control composition that does not contain the cationically modified poly alpha-1,6-glucan ether compound.

25. The personal care composition of paragraph 24, wherein the composition is substantially free of inorganic salt.

26. The personal care composition of paragraph 24, wherein the composition deposits at least 10% more of the silicone conditioning agent than the control composition, preferably at least 25%, more preferably at least 50%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
an anionic detersive surfactant;
an co-surfactant selected from non-ionic, amphoteric and zwitterionic surfactants;
a cationic polymer;
a cationically modified poly alpha-1,6-glucan ether compound; and
an aqueous carrier.

2. The personal care composition of claim 1, wherein the cationically modified poly alpha-1,6-glucan ether compound comprises a backbone of glucose monomer units, wherein at least 65% of the backbone glucose monomer units are linked via alpha-1,6-glycosidic linkages.

3. The personal care composition of claim 1, wherein the cationically modified poly alpha-1,6-glucan ether compound has a degree of substitution of 0.01 to 0.3.

4. The personal care composition of claim 1, wherein the cationically modified poly alpha-1,6-glucan ether compound has a weight average molecular weight of 50000 to 500,000 daltons.

5. The personal care composition of claim 1, wherein the cationically modified poly alpha-1,6-glucan ether compound has a charge density of 0.05 to 3.

6. The personal care composition of claim 1, wherein 5% to 35% of the backbone glucose monomer units have branches via alpha-1,2 and/or alpha-1,3-glycosidic linkages.

7. The personal care composition of claim 1, wherein the poly alpha-1,6-glucan ether compound comprises a cationically modified poly alpha-1,6-glucan substituted with at least one positively charged ammonium group.

8. The personal care composition of claim 7, wherein the substituted ammonium group comprises a quaternary ammonium group comprising at least one C10 to C16 alkyl group.

9. The personal care composition of claim 8, wherein the quaternary ammonium group comprises a trimethylammonium group.

10. The personal care composition of claim 1, wherein the cationically modified poly alpha-1,6-glucan ether compound improves at least one conditioning benefit selected from Wet Feel, Final Rinse Friction, Wet Detangling, Wet Combing, Dry Detangling, Dry Combing, Resistance at Tips, Smoothness and Force to Comb compared to a control composition that does not include the cationically modified poly alpha-1,6-glucan ether compound.

11. The personal care composition of claim 10, wherein the composition provides a better dilute coacervate than a control composition that does not include the cationically modified poly alpha-1,6-glucan ether compound, according to the % T method.

12. The personal care composition of claim 1, wherein the composition does not comprise an in-situ coacervate.

13. The personal care composition of claim 11, wherein the composition has a % T of greater than 80, preferably greater than 90, more preferably greater than 95.

14. The personal care composition of claim 1, wherein the anionic detersive surfactant is selected from alkyl sulfates, alkyl ether sulfates, acyl glycinates, acyl sarcosinates, acyl glutamates, acyl alaninates, sulfosuccinates, isethionates, sulfonates, sulfoacetates, glucose carboxylates, alkyl ether carboxylates, acyl taurates, sodium, ammonium or potassium salts of these, and mixtures thereof.

15. The personal care composition of claim 1, wherein the co-surfactant is selected from aliphatic secondary and tertiary amines, wherein at least one aliphatic substituent contains 8 to 18 carbon atoms and at least one aliphatic substituent contains an anionic selected from carboxy, sulfonate, sulfate, phosphate, and phosphonate.

16. The personal care composition of claim 14, wherein the co-surfactant is selected from ocoamphoacetate, cocoamphodiacetate, lauroamphoacetate, lauroamphodiacetate, and mixtures thereof.

17. The personal care composition of claim 1, wherein the co-surfactant is selected from derivatives of aliphatic quaternary ammonium, phosphonium, and sulfonium compounds, wherein at least one aliphatic substituent contains 8 to 18 carbon atoms and at least one aliphatic substituent contains an anionic group selected from carboxy, sulfonate, sulfate, phosphate and phosphonate.

18. A personal care composition, comprising:
an anionic detersive surfactant;
an amphoteric co-surfactant;
a cationically modified poly alpha-1,6-glucan ether compound; and
an aqueous carrier, wherein the composition is free of sulfated surfactants and exhibits improved stability compared to a control composition that does not contain the cationically modified poly alpha-1,6-glucan ether compound.

19. A personal care composition, comprising:
an anionic detersive surfactant;
an amphoteric co-surfactant;
a cationically modified poly alpha-1,6-glucan ether compound;
a silicone conditioning agent; and
an aqueous carrier, wherein the composition exhibits improved deposition of the silicone conditioning agent according to the Deposition test when compared to a control composition that does not contain the cationically modified poly alpha-1,6-glucan ether compound.

20. The personal care composition of claim 19, wherein the composition is substantially free of inorganic salt.
